(19) United States
(12) Patent Application Publication
(10) Pub. No.: US 2013/0103108 A1
(43) Pub. Date: Apr. 25, 2013

(54) METHOD AND APPARATUS TO INCREASE STROKE VOLUME BY SYNCHRONIZING / MODULATING HEART RATE WITH ACTIVITY RATE

(71) Applicants: Steve Koh, South Pasadena, CA (US); Gene A. Bornzin, Simi Valley, CA (US)

(72) Inventors: Steve Koh, South Pasadena, CA (US); Gene A. Bornzin, Simi Valley, CA (US)

(73) Assignee: PACESETTER, INC., Sylmar, CA (US)

(21) Appl. No.: 13/277,020

(22) Filed: Oct. 19, 2011

(51) Int. Cl. A61N 1/365

(52) U.S. Cl. 607/19

(57) ABSTRACT

A method of synchronizing a heart rate with an activity rate of a patient includes determining the activity rate of the patient. The method also includes synchronizing a pacing pulse with a phase of the activity rate to improve a cardiac stroke volume of the patient. The synchronizing includes lowering the heart rate during down motion associated with the activity rate and increasing the heart rate during an up motion associated with the activity rate when a stride rate is slower than a target heart rate.

### FIELD

The present disclosure is related, generally, to a method and system for increasing stroke volume, more specifically to a method and system for increasing stroke volume by synchronizing/modulating heart rate with body stride rate assessed via, for example, a three dimensional accelerometer.

### BACKGROUND

Arterial counter-pulsation has been used to increase stroke volume but it requires placing an inflatable tube within descending thoracic aorta. The same phenomenon can be observed during running/walking by a patient if heart rate and stride rate can be entrained and if appropriate phase can be achieved. For example, FIG. 3 shows exemplary graphs (e.g., standing waves) illustrating pressure pulse in an artery of twelve patients with respect to time when the heart and step rate (or stride rate) are entrained. For each patient 1-12, a pair of graphs indicates pressure pulse in phase and out of phase with the patient's movement respectively. In particular, the graphs are based on twelve patients subjected to entrainment/random heart-stride relation testing with a catherized pressure sensor.

The mean arterial pressure (MAP) for in-phase was determined to be 108±26 mmHg and for out of phase entrainment was 38±17 mmHg. MAP describes an average blood pressure in an individual, defined as the average arterial pressure during a single cardiac cycle. In general, MAP is normally between 70 to 110 mmHg and if MAP falls significantly below this number for an appreciable time, the end organ will not get enough blood flow, and will become ischemic. The graph illustrates that entrainment with appropriate phasing could mimic arterial counter-pulsation and create a favorable situation where heart motion and bodily motion are in unison. The benefits associated with entrainment include increase in stroke volume, enhanced oxygen delivery, less myocardial oxygen consumption due to less energy expenditure from the heart.

Therefore, it is desirable to provide a system or method which would provide the benefits of entrainment without the drawbacks of current solutions.

### SUMMARY

According to some aspects of the disclosure, a method of synchronizing a heart rate with an activity rate of a patient includes determining the activity rate of the patient. The method also includes synchronizing a pacing pulse with a phase of the activity rate to improve a cardiac stroke volume of the patient.

According to some aspects of the disclosure, an apparatus for synchronizing a heart rate with an activity rate of a patient includes a controller configured to determine the activity rate of the patient. The apparatus also includes a synchronization device configured to synchronize a pacing pulse with a phase of the activity rate to improve a cardiac stroke volume of the patient.

According to some aspects of the disclosure, an apparatus for synchronizing a heart rate with an activity rate of a patient includes means for determining the activity rate of the patient. The apparatus also includes means for synchronizing a pacing pulse with a phase of the activity rate to improve a cardiac stroke volume of the patient.

According to some aspects of the disclosure, an apparatus for synchronizing a heart rate with an activity rate of a patient includes a memory and one or more processors coupled to the memory. The processor(s) is configured to determine the activity rate of the patient. The processor(s) is further configured to synchronize a pacing pulse with a phase of the activity rate to improve a cardiac stroke volume of the patient.

According to some aspects of the disclosure, a computer program product for synchronizing a heart rate with an activity rate of a patient includes a computer-readable medium having non-transitory program code recorded thereon. The program code includes program code to determine the activity rate of the patient. The program code also includes program code to synchronize a pacing pulse with a phase of the activity rate to improve a cardiac stroke volume of the patient.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying FIGURES. It is to be expressly understood, however, that each of the FIGURES is provided for the purpose of illustration and description only and is not intended as a definition of the limits of the present disclosure.

### BRIEF DESCRIPTION OF FIGURES

The features, nature, and advantages of the present disclosure will become more apparent from the detailed description set forth below when taken in conjunction with the drawings in which like reference characters identify correspondingly throughout.

FIG. 1 schematically illustrates an exemplary implantable medical device (IMD) in electrical communication with the heart of a patient.

FIG. 2 schematically illustrates an exemplary implantable stimulation device configured as a system according to some aspects of the disclosure.

FIG. 3 illustrates a sample of pressure waves of twelve subjects/patients when the heart rate and step or motion rate of each patient are entrained.

FIG. 4 illustrates an exemplary heart rate synchronization system according to some aspects of the disclosure.

FIGS. 5A to 5D illustrate a coordinated variation of the relationship between the cardiac cycle and a patient's motion or step.

FIGS. 5E to 5H illustrate a relationship between a stride rate of a patient relative to a pacing interval according to aspects of the disclosure.

FIG. 6 illustrates a method of modulating heart rate based on up-down motion of a patient in order to increase stroke volume.

FIG. 7 illustrates a method for synchronizing heart rate and step rate of a patient according to some aspects of the disclosure.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion. The following description includes the best mode presently contemplated for practicing the present teachings. The description is not to be taken in a limiting sense but is merely for the purpose of describing the general principles of the illustrative embodiments. The scope of the present teachings should be ascertained with reference to the claims. In the description that follows, like numerals or reference designators will refer to like parts or elements throughout.

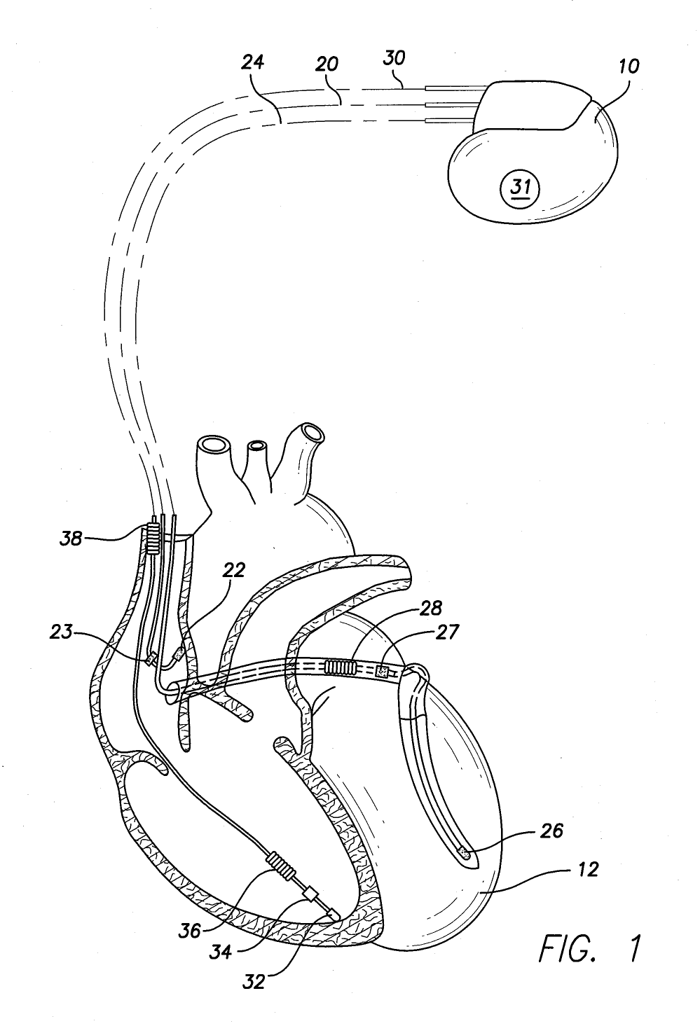

With reference to FIG. 1, an exemplary implantable medical device (IMD) will be described in detail. The IMD 10 is in electrical communication with the heart 12 of a patient by way of three leads, 20, 24 and 30, suitable for delivering multi-chamber stimulation and shock therapy. To sense atrial cardiac signals and to provide right atrial chamber stimulation therapy, the IMD 10 is coupled to an implantable right atrial lead 20 having at least an atrial tip electrode 22, which typically is implanted in the right atrial appendage, and an atrial ring electrode 23.

To sense left atrial and ventricular cardiac signals and to provide left chamber pacing therapy, the IMD 10 is coupled to a “coronary sinus” lead 24 designed for placement in the “coronary sinus region” via the coronary sinus or for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28.

The IMD 10 is also shown in electrical communication with the heart by way of an implantable right ventricular lead (RV lead) 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart so as to place the right ventricular tip electrode 32 in the right ventricular apex so the RV coil electrode 36 is positioned in the right ventricle and the SVC coil electrode 38 is positioned in the superior vena cava. Accordingly, the right ventricular lead 30 is capable of receiving cardiac signals, and delivering stimulation in the form of pacing and shock therapy to the right ventricle. To provide a “vibratory alert” signal (from a motor with an offset mass that can be provided in the device can), an additional electrode can be provided in proximity to the device can. An accelerometer 31 can also be provided to sense three dimensional movements.

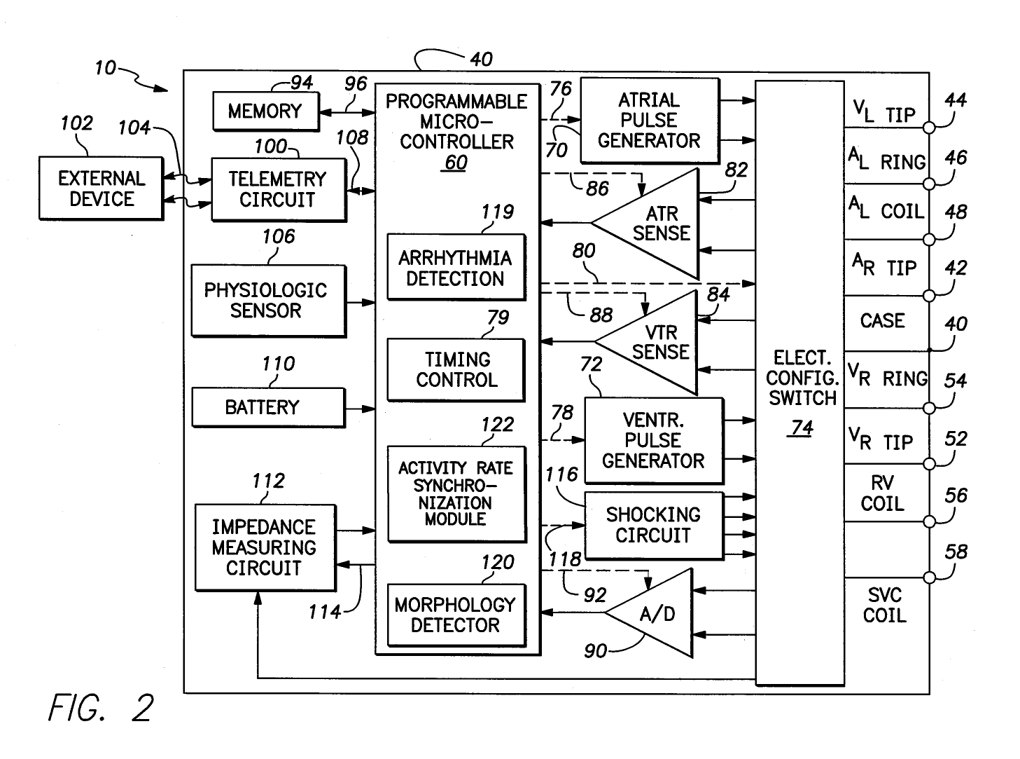

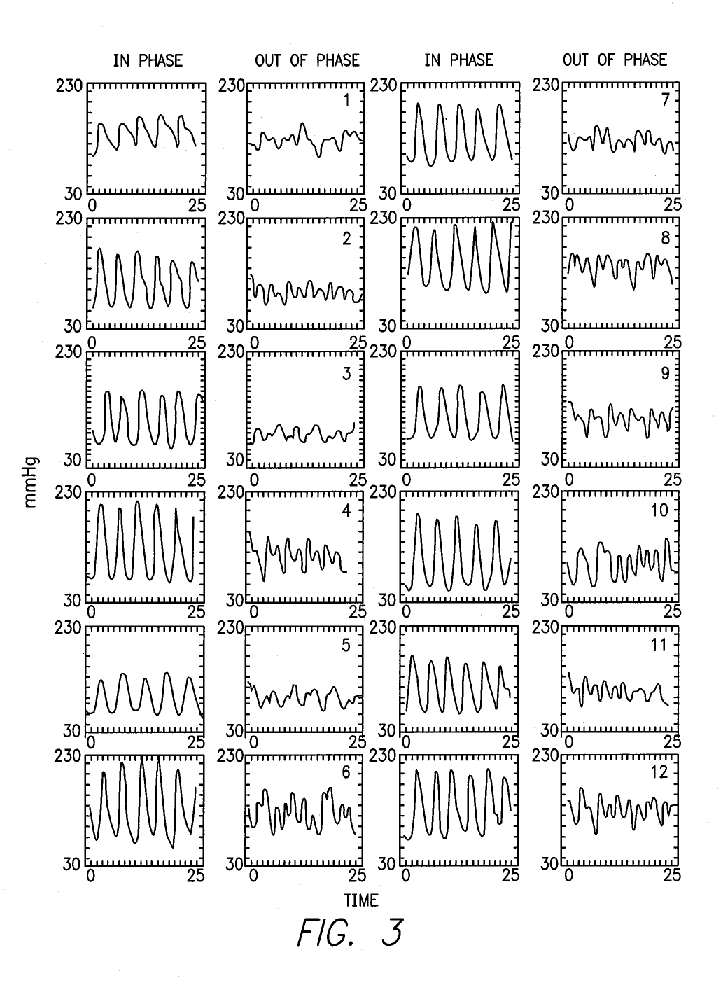

As illustrated in FIG. 2, a simplified block diagram is shown of the multi-chamber implantable IMD 10, which is capable of treating both fast and slow arrhythmias with stimulation therapy, including cardioversion, defibrillation, and pacing stimulation. The IMD 10 is configured as a system in which the various embodiments of the present teachings may operate. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

The housing 40 for the IMD 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes, 28, 36 and 38, (FIG. 1) for shocking purposes. The housing 40 further includes a connector (not shown) having multiple terminals, 42, 44, 46, 48, 52, 54, 56 and 58 (shown schematically and, for convenience, the names of the electrodes to which they are connected are shown next to the terminals).

As such, to achieve right atrial sensing and pacing, the connector includes at least a right atrial tip terminal (AR TIP) 42 adapted for connection to the atrial tip electrode 22 (FIG. 1) and a right atrial ring (AR RING) electrode (not shown) adapted for connection to the right atrial ring electrode 23 (FIG. 1). To achieve left chamber sensing, pacing and shocking, the connector includes at least a left ventricular tip terminal (VL TIP) 44, a left atrial ring terminal (AL RING) 46, and a left atrial shocking terminal (AL COIL) 48, which are adapted for connection to the left ventricular ring electrode 26 (FIG. 1), the left atrial tip electrode 27 (FIG. 1), and the left atrial coil electrode 28 (FIG. 1), respectively. To support right chamber sensing, pacing and shocking, the connector further includes a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32 (FIG. 1), right ventricular ring electrode 34 (FIG. 1), the RV coil electrode 36 (FIG. 1), and the SVC coil electrode 38 (FIG. 1), respectively. To provide the “vibratory alert” signal, a vibratory alert unit (not shown) generates a signal for an additional terminal (not shown) for connection to the vibratory alert electrode. In one embodiment, the vibratory alert will alert the patient, and then a home monitor can be used to transfer the information associated with the alert from the device 10 to an attending medical professional, who can take the appropriate clinical action.

The IMD 10 includes a programmable microcontroller 60, which controls the various modes of stimulation therapy. As is well known in the art, the microcontroller 60 (also referred to as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by program code stored in a designated block of the memory. The details of the design and operation of the microcontroller 60 are not critical to the present teachings. Rather, any suitable microcontroller 60 may be used that carries out the functions described. The use of microprocessor-based control circuits for performing timing and data analysis functions is well known in the art.

As shown in FIG. 2, an atrial pulse generator 70 and a ventricular pulse generator 72 generate pacing stimulation pulses for delivery by the right atrial lead 20 (FIG. 1), the right ventricular lead 30 (FIG. 1), and/or the coronary sinus lead 24 (FIG. 1) via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators or shared pulse generators. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 that controls the timing of such stimulation pulses (such as pacing rate, atrio-ventricular (AV) delay, atrial interconduction (A-A) delay, or ventricular interconduction (V-V) delay, and the like) as well as to keep track of the timing of refractory periods, blanking intervals, noise detection windows, evoked response windows, alert intervals, marker channel timing, etc., as is well known in the art. A switch 74 includes multiple switches for connecting the desired electrodes to the appropriate I/O circuits, thereby providing complete electrode programmability. Accordingly, the switch 74, in response to a control signal 80 from the microcontroller 60, determines the polarity of the stimulation pulses (such as unipolar, bipolar, combipolar, etc.) by selectively closing the appropriate combination of switches (not shown) as is known in the art.

Atrial sensing circuits 82 and ventricular sensing circuits 84 may also be selectively coupled to the right atrial lead 20 (FIG. 1), the coronary sinus lead 24 (FIG. 1), and the right ventricular lead 30 (FIG. 1), through the switch 74 for detecting the presence of cardiac activity in each of the four chambers of the heart. Accordingly, the atrial (ATR. SENSE) and ventricular (VTR. SENSE) sensing circuits, 82 and 84, may include dedicated sense amplifiers, multiplexed amplifiers or shared amplifiers and may receive control signals 86, 88 from the controller 60. The switch 74 determines the “sensing polarity” of the cardiac signal by selectively closing the appropriate switches, as is also known in the art. In this way, the clinician may program the sensing polarity independent of the stimulation polarity.

Each sensing circuit, 82 and 84, employs one or more low power, precision amplifiers with programmable gain and/or automatic gain control, band pass filtering, and a threshold detection circuit, as known in the art, to selectively sense the cardiac signal of interest. An automatic gain control enables the device 10 to effectively address the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (for example: P-waves, R-waves, and depolarization signals associated with fibrillation which are sometimes referred to as “F-waves” or “Fib-waves”) are then classified by the microcontroller 60 by comparing them to a predefined rate zone limit (for example: bradycardia, normal, low rate VT, high rate VT, and fibrillation rate zones) and various other characteristics (for example: sudden onset, stability, physiologic sensors, and morphology, and the like) in order to determine the type of remedial therapy that is needed (for example: bradycardia pacing, anti-tachycardia pacing, cardioversion shocks or defibrillation shocks, and the like).

Cardiac signals are also applied to the inputs of an analog-to-digital (A/D) data acquisition system 90. The data acquisition system 90 is configured to acquire intra-cardiac electrogram (IEGM) signals, convert the raw analog data into a digital signal, and store the digital signals for later processing and/or telemetric transmission to an external device 102. The data acquisition system 90 is coupled to the right atrial lead 20 (FIG. 1), the coronary sinus lead 24 (FIG. 1), and the right ventricular lead 30 (FIG. 1) through the switch 74 to sample cardiac signals across any pair of desired electrodes. The controller 60 controls the data acquisition system via control signals 92. Although FIG. 1 depicts a coronary sinus lead 24, an intrapericardial lead can replace or augment the coronary sinus lead 24.

The microcontroller 60 is further coupled to a memory 94 by a suitable data/address bus 96. The programmable operating parameters used by the microcontroller 60 are stored and modified, as required, in order to customize the operation of the IMD 10 to suit the needs of a particular patient. The memory 94 includes software modules, such as an activity rate synchronizer 122, which, when executed or used by the microcontroller 60, provide the operational functions of the IMD 10. Additional operating parameters and code stored on the memory 94 define, for example, a pacing pulse amplitude or magnitude, pulse duration, electrode polarity, rate, sensitivity, automatic features, arrhythmia detection criteria, and the amplitude, wave shape and vector of each shocking pulse to be delivered to the patient's heart within each respective tier of therapy. Other pacing parameters include base rate, rest rate and circadian base rate.

Advantageously, the operating parameters of the implantable medical device 10 may be non-invasively programmed into the memory 94 through a telemetry circuit 100 in telemetric communication with the external device 102, such as a programmer, trans-telephonic transceiver, a diagnostic system analyzer, or even a cellular telephone. The telemetry circuit 100 is activated by the microcontroller by a control signal 108. The telemetry circuit 100 advantageously allows intra-cardiac electrograms and status information relating to the operation of the device 10 (as contained in the microcontroller 60 or memory 94) to be sent to the external device 102 through an established communication link 104. In one embodiment, the IMD 10 further includes a physiologic sensor 106, commonly referred to as a “rate-responsive” sensor because it adjusts pacing stimulation rate according to the exercise state of the patient. However, the physiological sensor 106 may further be used to detect changes in cardiac output, changes in the physiological condition of the heart, or diurnal changes in activity (for example, detecting sleep and wake states). Accordingly, the microcontroller 60 responds by adjusting the various pacing parameters (such as rate, AV Delay, V-V Delay, and others) at which the atrial and ventricular pulse generators, 70 and 72, generate stimulation pulses. While shown as being included within the IMD 10, it is to be understood that the physiologic sensor 106 may also be external to the IMD 10, yet still be implanted within or carried by the patient.

The IMD 10 additionally includes a battery 110, which provides operating power to all of the circuits shown in FIG. 2. For the IMD 10, which employs shocking therapy, the battery 110 is capable of operating at low current drains for long periods of time, and is capable of providing high-current pulses (for example, for capacitor charging) when the patient requires a shock pulse. The battery 110 also has a predictable discharge characteristic so that elective replacement time can be detected. In one embodiment, the device 10 employs lithium/silver vanadium oxide batteries. As further shown in FIG. 2, the device 10 has an impedance measuring circuit 112 enabled by the microcontroller 60 via a control signal 114.

The IMD 10 detects the occurrence of an arrhythmia and automatically applies an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 or more joules), as controlled by the microcontroller 60. Such shocking pulses are applied to the heart 12 through at least two shocking electrodes, and as shown in this embodiment, selected from the left atrial coil electrode 28 (FIG. 1), the RV coil electrode 36 (FIG. 1), and/or the SVC coil electrode 38 (FIG. 1). As noted above, the housing 40 may function as an active electrode in combination with the RV coil electrode 36 (FIG. 1), or as part of a split electrical vector using the SVC coil electrode 38 (FIG. 1) or the left atrial coil electrode 28 (FIG. 1) (for example, by using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (such as corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

The microcontroller 60 includes a morphology detector 120 for tracking various morphological features within electrical cardiac signals, including intervals between polarization events, elevations between polarization events, durations of polarization events and amplitudes of polarization events. The microcontroller 60 also includes an arrhythmia detection control 119 that analyzes the sensed electrical signals to determine whether or not arrhythmia is being experienced. An activity rate synchronizer module 122, in cooperation with the memory 94, assists in synchronizing the activity rate with the heart rate, as discussed in more detail below.

The remaining FIGURES, flow charts, graphs and other diagrams illustrate the operation and novel features of the IMD 10 as configured in accordance with exemplary embodiments of the present teachings. In the flow chart, the various process steps are summarized in individual “blocks.” Such blocks describe specific actions or decisions made or carried out as the process proceeds. Where a microcontroller (or equivalent) is employed, the flow chart provides the basis for activity rate synchronizing processing that may be used by such a microcontroller (or equivalent IMD controller) to adaptively synchronize an activity rate with a heart rate. Those skilled in the art may readily write such a program based on the flow chart and other descriptions presented herein.

In some aspects, a three dimensional (3D) sensor 31, e.g., three dimensional accelerometer, can be used to accomplish similar benefits as those accomplished by entrainment. The three dimensional accelerometer may be configured to measure activity based on acceleration of the patient. The three dimensional sensor or accelerometer may be configured to detect the position of a patient, e.g., upright position, and the orientation of the patient, e.g., x, y or z-axis. A pacing scheme based on patient activity/gait assessment can be established in conjunction with the three dimensional accelerometer. In this implementation, it is desirable to establish a synchronized functional relationship between the heart rate and an activity rate, e.g., a stride rate, of a patient that is based on external motion of the patient. In some aspects, the implementation is configured to decrease or eliminate the undesirable or unfavorable relationships between the pulse waveform associated with the artery and the waveform representing the patient's motion and to enhance the favorable effects.

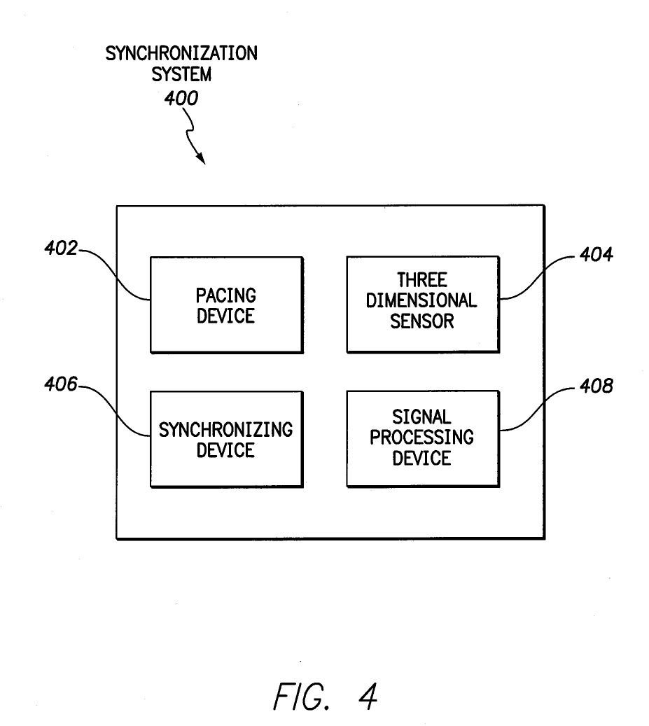

FIG. 4 illustrates system for increasing stroke volume according to some aspects of the disclosure. The activity rate synchronization system 400 includes a three dimensional sensor 404 (such as the sensor 31 of FIG. 1), for example, a three dimensional accelerometer, a synchronization device 406, a processing device 408 and a pacing device 402. In some aspects, the pacing device 402 may be an independent pacing device or may be integrated in the IMD 10 or an external heart monitoring system. The pacing device 402 may be configured to transmit pacing signals to leads 20, 24 and 30 or alternatively to an intrapericardial lead. In some aspects, the system 400 may be configured to discern the acceleration of blood in the thorax that corresponds to the highest degree of acceleration of the patient's motion and to synchronize the patient's heart beat with this acceleration and with pacing signals in order to increase the stroke volume.

The three dimensional movement of a patient produced by activities such as walking, running, jumping etc., can be recorded by the three dimensional accelerometer 404. In some aspects, the activities/motions of the patient can be categorized as an x-axis acceleration or motion (e.g., forward-backward acceleration, a z-axis acceleration (e.g., left-right acceleration) and a y-axis acceleration (e.g., vertical or up-down acceleration). The three dimensional accelerometer 404 may be configured (possibly in conjunction with a processor) to establish the axis that corresponds to increased acceleration of blood in the chest with a foot plant or other event associated with running or walking, for example.

The activity sensed or measured by the three dimensional accelerometer 404 can be processed by the processing device 408 using frequency domain methods and filtering techniques. In some aspects, the three dimensional sensor 404 may sense movement of the patient and determine the activity or stride that is dominant and the axis of such dominant activity. In some aspects, this determination of the three dimensional accelerometer may be accomplished in conjunction with a processor, e.g., processing device 408. The processor may be internal or external to the three dimensional sensor 404. The activity can be represented as a dominant vector. The dominant activity or stride may correspond to the activity or stride with fastest acceleration or activity, for example. For example, the dominant activity may correspond to an “up” motion of a patient's “up-down” motion along the y-axis.

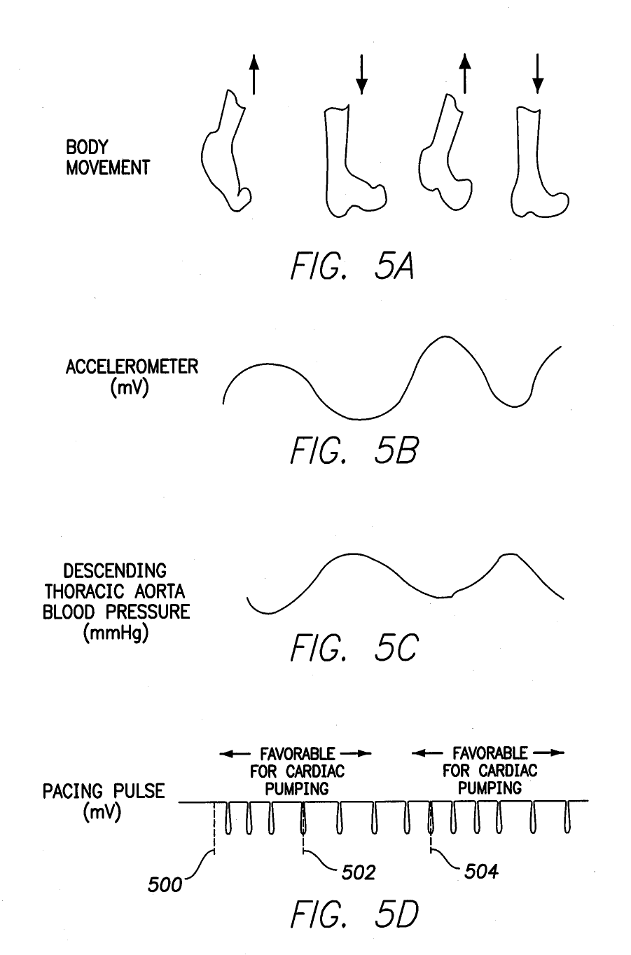

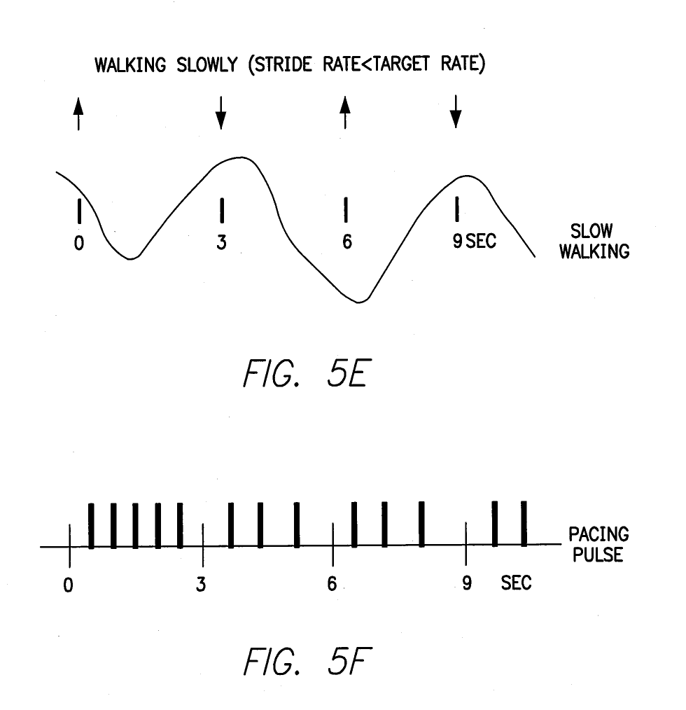

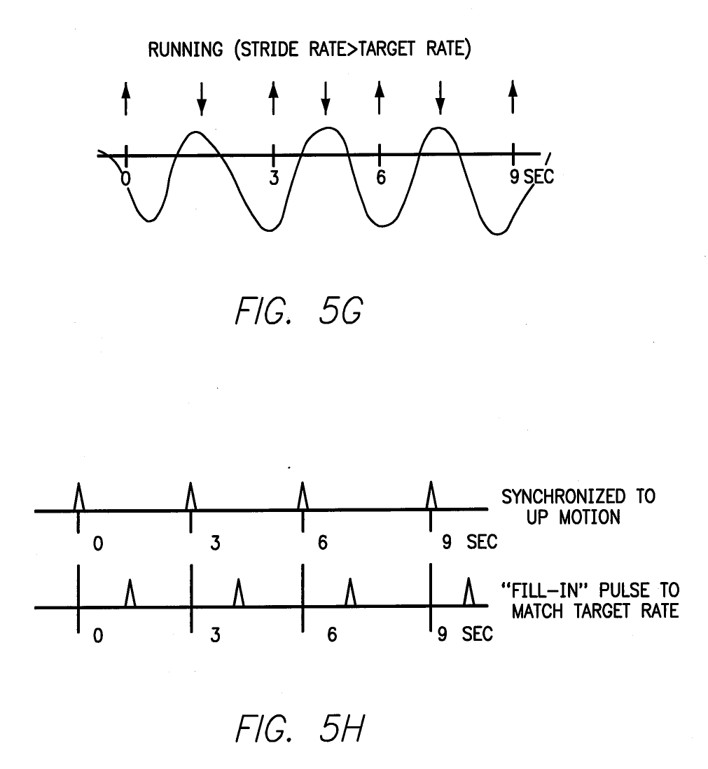

The three dimensional sensor 404, e.g., three dimensional accelerometer, senses motion of the patient, e.g., up/down motion (see FIG. 5A), and generates an activity signal representing the motion. The three dimensional motion can include anterior/posterior motion, an up/down motion or a combination. In some aspects, the activity signal representing an up/down motion is received at the processing device 408 where the signal may be filtered by a high pass filter, for example, and the frequency response obtained. In some aspects, the frequency response of the activity signal may be in the range of 1-20 Hz. The activity signal can be rectified and integrated to generate a mean of the activity signal. In some aspects, a processor, e.g., the processing device 408, associated with the three dimensional sensor 404, samples the activity signal that is integrated over one second to develop a periodic pattern, e.g., stride rate, of the activity signal. In one configuration, the activity signal is propagated in blocks of one second.

In some aspects, it is desirable to synchronize the periodic pattern of the activity signal with the heart rate of the patient. This feature can be accomplished by developing a functional relationship between the periodic pattern, e.g., stride rate, and the heart rate of the patient. The functional relationship may be based on monitoring the phase or rate of the patient's activity or motion and the phase or rate of the patient's heartbeat. In some aspects, the activity signal may be filtered digitally (as noted above), and the result multiplied by a constant to establish the heart rate.

The three dimensional sensor 404 may be incorporated in the implantable medical device (IMD) or be external to the IMD 10. In some aspects, the three dimensional sensor may be incorporated in the leads 20, 24 and 30 or alternatively in an intrapericardial lead. The external heart monitoring device and/or the implantable medical device may be configured to receive or acquire signals (e.g., heart activity signals) from or to transmit signals, (e.g., pacing signals), to the leads 20, 24, 30 and/or the intrapericardial lead. Signals from the three dimensional sensor 404 can be received at the microcontroller 60 of the implantable medical device 10 or a similar processor at the external heart monitoring device.

In some aspects, when the three dimensional sensor 404 detects an increased/decreased dominant activity from the patient, the three dimensional sensor 404 initiates a pattern analysis to determine the axis of the motion, e.g., y-axis (up-down acceleration). The pattern analysis may yield a periodic pattern of the motion of the patient. The periodic pattern may yield the stride rate of the patient moving along the y-axis. In some aspects the pattern analysis may be initiated or performed by a processor coupled to the three dimensional sensor 404, (e.g., processing device 408).

The pacing device 402 may include or be associated with a physiological sensor 106 configured to determine the heart rate of the patient. The heart rate may be determined according to the heart sounds sensed by the physiological sensors and detected by processors associated with the physiological sensor. The pacing device 402 can adjust its pacing rate based on sensing the patient's intrinsic atrial activity and regulating, for example, the ventricular pacing rate accordingly. The adjustment of the pacing rate may result in an increase or a decrease of the heart rate.

In some aspects, dual chamber pacing can be implemented to establish a target heart rate in synchronization with the dominant acceleration of the patient. In some aspects, when the stride rate is similar to the target heart rate, the synchronizing device 406 can synchronize pacing with the time of a dominant stride of the patient. The dominant stride may correspond to the ‘up’ motion of the patient's stride, for example. The pacing device 402 may be configured to send pacing signals to the heart to vary the heart rate in order to synchronize the heart rate with the stride rate.

Aspects of the disclosure increase stroke volume. During ‘up’ motion, for example, the thoracic arterial blood accelerates to flow down (due to action-reaction) which in turn relieves pressure in the aortic/great vessel artery. When the stride rate is slower than a target heart rate, the pacing device 402 may adjust the pacing rate such that the heart rate is lowered during a “down” motion, for example, which may increase the thoracic arterial blood pressure, but the heart rate is increased during “up” motion. Thus, a pacing rate or an interval between a pacing pulse can be adjusted based on the up/down motion. For example, during the up motion, the pacing rate is increased to accommodate the drop in blood pressure associated with the up motion. Similarly, during the down motion, the pacing rate is decreased to accommodate an increase in blood pressure. The target heart rate can be dynamically determined based on signals from an activity sensor or can be pre-programmed, for example by a physician, as a fixed value.

FIG. 5A through 5D illustrate a coordinated relationship between the cardiac cycle and the patient's motion. As the patient moves up and down as illustrated in FIG. 5A, the three dimensional accelerometer 404 senses the motion of the patient and generates a signal, e.g., accelerometer signal, illustrated in FIG. 5B that represents the up and down motion or acceleration of the patient. FIG. 5B illustrates a graph of a voltage pulse or signal representing the patient's motion, with voltage on the y-axis and time on the x-axis. The crest of the signal generated represents the up motion of the patient in the y-axis and the bottom of the signal represents the downward motion of the patient. FIG. 5C illustrates a signal representing the pressure pulse in an artery, e.g., aorta, with time on the x-axis and pressure on the y-axis.

A favorable or desirable cardiac pumping scenario occurs when the pressure pulse generated by the heart beat and the surge induced by the patient's up-down motion, are out of phase by 180 degrees, for example. In this scenario, the change in pressure within the aorta (e.g., descending aorta) related to the patient's movement is out of phase with the cardiac ejection. Therefore, a chamber of the heart relaxes when blood rushes into the chamber during an up motion of the patient and contracts to eject blood out of the chamber during a down motion of the patient. This synchronized implementation increases the stroke volume. In order to further increase the stroke volume, it is desirable to vary the pacing pulses in the out of phase scenario as illustrated in FIG. 5D in order to improve the functional relationship between the heart rate and the stride rate of the patient. In some aspects, when the target heart rate is less than or equal to the stride or activity rate of a pacing pulse or increased pacing pulse, the heart rate is synchronized with the “up” motion in order to increase or maximize stroke volume. For example, in the up motion, i.e., interval between times 500 and 502, the pacing rate is increased to accommodate a drop in blood pressure associated with the up motion. Similarly, in the down motion, i.e. interval between times 502 and 504, the pacing rate is decreased to accommodate an increase in blood pressure. In FIG. 5D, the interval between times 500 and 502 is shorter than the interval between times 502 and 504. Thus, although three pulses appear in each interval, the rate is actually higher in the first interval.

It is also desirable to establish whether cardiac output is enhanced. In some aspects, intra-cardiac and extra-cardiac vectors or parameters can be useful in discerning stroke volume. This feature can be accomplished based on a measurement of impedance factors associated with the cardiac cycle. In some aspects, the impedance factors can be measured by the impedance measuring circuit 112. In some aspects, the effects of heart rate modulation may be assessed based on superior vena cava (SVC) to pacemaker case impedance or based on the measurement by a pressure sensor. In some aspects, the impedance may be based on surges of blood leaving the heart. The impedance changes may occur with every heartbeat when the impedance decreases because of blood leaving the heart.

Cardiac output information based on impedance determination, for example, may be useful to establish the precise relationship between the heart rate and the stride rate to improve or optimize the stroke volume. Cardiac output information may also be useful to discern that blood ejection was more effective at a given instance, phase or point in time. This information can be helpful to determine when to pace relative to the activity or foot plant, for example. The information may result in pacing before a patient's acceleration or at the acceleration, for example.

FIGS. 5E to 5H illustrate a relationship between a stride rate of a patient and a pacing interval according to some aspects of the disclosure. In particular, FIGS. 5E and 5G illustrate a signal representing the pressure pulse in an artery or chamber of the patient, e.g., aorta, with time on the x-axis and pressure on the y-axis. FIGS. 5F and 5H illustrate a pacing pulse administered to the patient at variable pacing intervals. With respect to FIGS. 5E and 5F, when the patient is walking slowly, the stride rate of the patient is less than the target heart rate. Variable pacing intervals may be applied based on whether the patient is in the up motion or the down motion. For example, when the patient is in the up motion (e.g., between 0 and 3 seconds) the pacing interval is faster and when the patient is in the down motion (e.g., between 3 and 6 seconds) the pacing interval is slower.

With respect to FIGS. 5G and 5H, when the patient is moving fast, e.g., running, the stride rate is greater than the target heart rate, and varying the pacing interval may not be desirable. FIG. 5H illustrate two sets of pacing pulses. In one configuration, synchronizing the pacing pulse to the up motion may be desirable. For example, when the patient runs at 70 paces/min and the target heart rate is 60, the IMD 10 can send pulses synchronized to the up motion with some pause to correspond to 60 bpm (beats per minute). In another configuration, “fill in” pulses may be introduced to match the target heart rate.

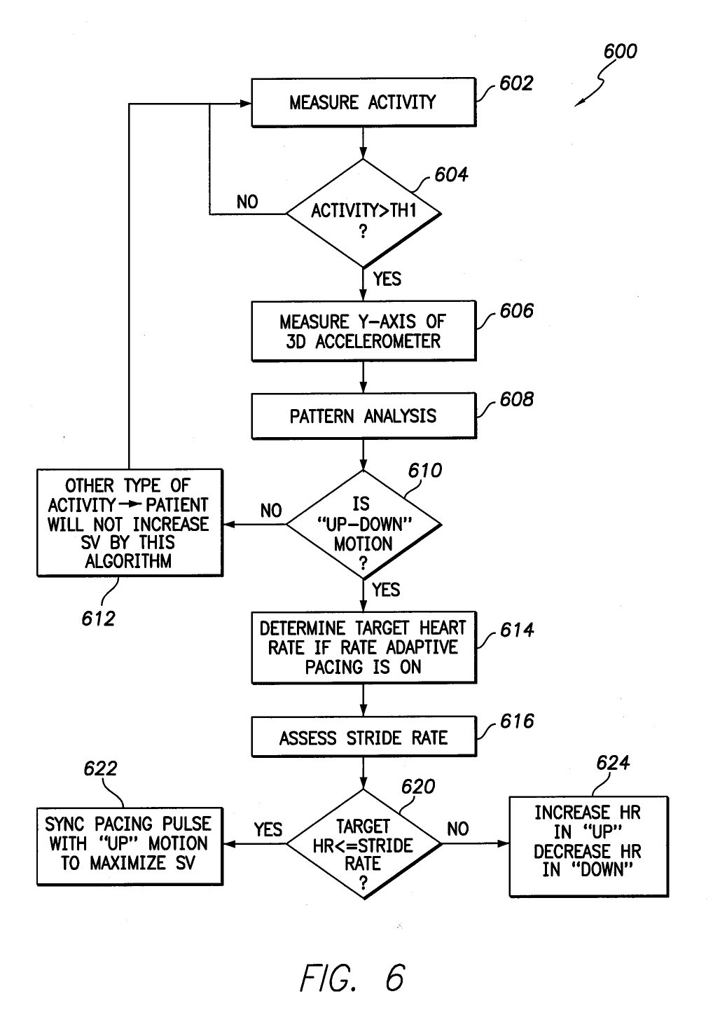

FIG. 6 illustrates a method or process 600 of modulating heart rate based on up-down motion of a patient in order to increase stroke volume. The method can be implemented in the activity rate synchronization system 400 illustrated in FIG. 4. The process begins at block 602 where the activity or motion of the patient is measured or sensed. The patient's motion can be sensed by the three dimensional accelerometer or sensor 404. At block 604, it is determined whether the patient's activity meets a threshold activity value. If the patients activity or motion meets the threshold activity value the process continues to block 606. Otherwise, the process returns to block 602. At block 606, a dominant motion, activity or stride of the patient is determined or measured by the three dimensional accelerometer, for example. In some aspects, the dominant stride or activity corresponds to the y-axis motion. At block 608, a pattern analysis of the patient's dominant activity is performed to determine a periodicity of the patient's motion. The patients stride, motion or activity rate can be determined based on the pattern analysis of the dominant activity.

The process continues to block 610 where it is determined whether the dominant activity is an “up-down” motion or activity of the patient. It can also be determined whether the periodicity is in an acceptable range for augmenting the heart. If the activity is an up-down activity and in an acceptable range, the process continues to block 614. Otherwise, the process continues to block 612 where the activity is indicated as an activity that would not increase stroke volume by synchronizing and the process subsequently returns to block 602. At block 614, a target heart rate of the patient is determined. The target heart rate may be determined based on an adaptive pacing implementation and/or a based on a programmed rate. If adaptive pacing is not active, a programmed rate will be used.

At block 616, the stride rate or the activity rate is assessed. The process continues to block 620 where it is determined whether the target heart rate is less than or equal to the stride or activity rate. If the target heart rate is less than or equal to the stride or activity rate, the process continues to block 622 where a pacing pulse is synchronized with the “up” motion in order to increase or maximize stroke volume. Otherwise, the process continues to block 624 where the heart rate is increased during “up” motion and decreased during “down” motion.

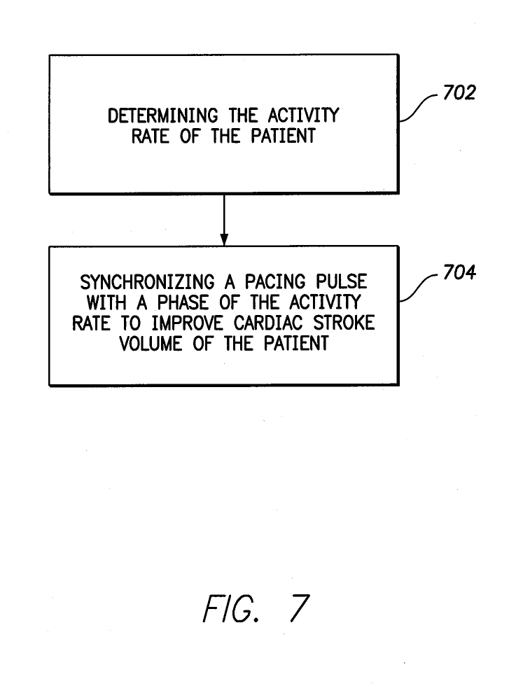

FIG. 7 illustrates a method for synchronizing heart rate and stride rate of a patient according to some aspects of the disclosure. At block 702, the method starts with determining an activity rate of the patient. At block 704, the method includes synchronizing a pacing pulse with a phase of the activity rate to improve a cardiac stroke volume of the patient.

In one configuration, the apparatus for synchronizing a heart rate with an activity rate of a patient includes means for determining the activity rate of the patient. In one aspect of the disclosure, the determining means may be the microcontroller 60, the activity rate synchronization system 400 and/or the three dimensional sensor 404 configured to perform the functions recited by the determining means. The apparatus is also configured to include means for synchronizing a pacing pulse with a phase of the activity rate to improve a cardiac stroke volume of the patient. In one aspect of the disclosure, the synchronizing means may be the synchronizing device 406, the activity rate synchronizer 122, the microcontroller 60 and/or the activity rate synchronization system 400 configured to perform the functions recited by the synchronizing means.

The methodologies described herein may be implemented by various means depending upon the application. For example, these methodologies may be implemented in hardware, firmware, software, or any combination thereof. For a hardware implementation, the processing units, including programmable microcontroller 60 (FIG. 2) may be implemented within one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, electronic devices, other electronic units designed to perform the functions described herein, or a combination thereof.

For a firmware and/or software implementation, the methodologies may be implemented with modules (e.g., procedures, functions, and so on) that perform the functions described herein. Any machine or computer readable medium tangibly embodying instructions that may be in a form implantable or coupled to an implantable medical device may be used in implementing the methodologies described herein. For example, software code may be stored in a memory and executed by a processor. When executed by the processor, the executing software code generates the operational environment that implements the various methodologies and functionalities of the different aspects of the teachings presented herein. Memory may be implemented within the processor or external to the processor. As used herein the term “memory” refers to any type of long term, short term, volatile, nonvolatile, or other memory and is not to be limited to any particular type of memory or number of memories, or type of media upon which memory is stored.

The machine or computer readable medium that stores the software code defining the methodologies and functions described herein includes physical computer storage media. A storage medium may be any available medium that can be accessed by the processor of an implantable medical device. By way of example, and not limitation, such computer-readable media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. As used herein, disk and/or disc includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer readable media.

Although the present teachings and its advantages have been described in detail, it should be understood that various changes, substitutions and alterations can be made herein without departing from the spirit and scope of the present teachings as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present teachings, processes, machines, manufacture, compositions of matter, means, methods, or steps, presently existing or later to be developed that perform substantially the same function or achieve substantially the same result as the corresponding embodiments described herein may be utilized according to the present teachings. Accordingly, the appended claims are intended to include within their scope such processes, machines, manufacture, compositions of matter, means, methods, or steps.

What is claimed is:

1. In an implantable cardiac stimulation device, a method of synchronizing a heart rate with activity of a patient, the method comprising:
detecting activity of the patient and one or more phases of the activity; and
synchronizing a pacing pulse with at least one of the phases of the activity to improve a cardiac stroke volume of the patient.

2. The method of claim 1, in which synchronizing comprises synchronizing the pacing pulse with a downward acceleration of the patient, based on the activity.

3. The method of claim 1, in which the synchronizing comprises lowering the heart rate during down motion associated with the activity and increasing the heart rate during an up motion associated with the activity when a stride rate is slower than a target heart rate.

4. The method of claim 1, in which determining the activity comprises assessing an axis of patient motion and determining whether the patient motion is periodic, and
the synchronizing comprises synchronizing only when the axis of patient motion corresponds to periodic up-down motion of the patient.

5. The method of claim 1, further comprising:
estimating stroke volume after the synchronizing; and
adjusting the synchronizing based on the estimated stroke volume.

6. An implantable cardiac stimulation apparatus for synchronizing a heart rate with an activity phase of a patient comprising:
a controller configured to detect motion of the patient in a predetermined direction; and
a synchronization device configured to synchronize a pacing pulse with the motion of the patient in the predetermined direction to improve a cardiac stroke volume of the patient.

7. The apparatus of claim 6, in which the synchronization device is further configured to synchronize the pacing pulse with downward acceleration of the patient, based on the activity.

8. The apparatus of claim 6, in which the synchronization device is further configured to synchronize by lowering the heart rate during a down motion associated with the activity and increasing the heart rate during an up motion associated with the activity when a stride rate is slower than a target heart rate.

9. The apparatus of claim 6, in which the controller is further configured to determine the activity by assessing an axis of patient motion and determining whether the patient motion is periodic.

10. The apparatus of claim 9, in which the synchronization device is further configured to synchronize only an axis of the patient motion that corresponds to periodic up-down motion of the patient.

11. An apparatus for synchronizing a heart rate with an activity rate of a patient, comprising:
means for determining the activity rate of the patient and motion of the patient in a predetermined direction; and
means for synchronizing a pacing pulse with the motion of the patient in the predetermined direction to improve a cardiac stroke volume of the patient.

12. The apparatus of claim 11, in which the synchronizing means comprises means for synchronizing the pacing pulse with downward acceleration of the patient, based on the activity rate.

13. The apparatus of claim 11, in which the synchronizing means comprises means for lowering the heart rate during down motion associated with the activity rate and increasing the heart rate during an up motion associated with the activity rate when a stride rate is slower than a target heart rate.

14. The apparatus of claim 11, in which the determining means comprises means for assessing an axis of patient motion and means for determining whether the patient motion is periodic; and
the synchronizing means comprises means for synchronizing only when the axis of patient motion corresponds to periodic up-down motion of the patient.

15. The apparatus of claim 11, further comprising:
means for estimating stroke volume after the synchronizing; and
means for adjusting the synchronizing based on the estimated stroke volume.
